SEQUENCE LISTING (3 sequences)

SEQ ID NO: 1 - protein, 49 aa, Artificial
  Full Chimeric Protein Sequence
NSYPGCPSSYDGYCLNGGVCMHIESLDSYTCNCVIGYSGDRCEHADLLA

SEQ ID NO: 2 - protein, 42 aa, Mus sp.
NSYPGCPSSYDGYCLNGGVCMHIESLDSYTCNCVIGYSGDRC

SEQ ID NO: 3 - protein, 7 aa, Mus sp.
EHADLLA

(19) United States
(12) Patent Application Publication
(10) Pub. No.: US 2008/0070838 A1
(43) Pub. Date: Mar. 20, 2008

(54) Growth Factor Treatment for Asthma

(75) Inventors: Donna Elizabeth Davies, Salisbury (GB); Stephen Holgate, Romsey (GB); Lynnsey M. Hamilton, Southampton (GB); Sarah Margaret Puddicombe, Southampton (GB); Audrey Richter, Fareham (GB)

Correspondence Address: MORRISON & FOERSTER LLP, 12531 HIGH BLUFF DRIVE, SUITE 100, SAN DIEGO, CA, 92130-2040, US

(73) Assignee: University of Southampton, Southampton, Hampshire (GB)

(21) Appl. No.: 11/575,061

(22) PCT Filed: Sep. 13, 2005
(86) PCT No.: PCT/GB05/50155

§ 371 Date: Nov. 21, 2007

(30) Foreign Application Priority Data
Sep. 13, 2004 (GB) 0420265.1
Nov. 17, 2004 (GB) 0425281.3

(51) Int. Cl. A61K 38/18; A61P 11/06; C12Q 1/02; C12Q 1/68

(52) U.S. Cl. 514/12; 435/29; 435/6

(57) ABSTRACT

The present invention relates to use of epidermal growth factor (EGF) analogues to treat, or protect from, bronchial epithelium damage in asthma patients. Suitable such analogues target the EGF receptor and exhibit ability to promote in asthma patients preferential proliferation of bronchial epithelial cells compared to airway fibroblasts.

The present invention relates to a new strategy for therapeutic intervention in relation to asthma. In particular, it relates to use of certain epithelial growth factor (EGF) analogues, to target, or protect from, bronchial epithelial damage in asthmatic patients. It also relates to alternative use of keratinocyte growth factor (KGF) or KGF analogues for the same therapeutic purpose.

### BACKGROUND TO THE INVENTION

Asthma is a chronic inflammatory disorder of the airways in which the airways constrict in response to common environmental factors such as allergens (e.g. house dust mites), viral infections and air pollutants resulting in breathlessness, wheeze and cough. The disease is progressive with repeated inflammatory damage to the epithelial lining of the airways and structural alternations (re-modelling) to the airway walls (Holgate, S. T. (1999) J. Allergy Clin. Immunol. 104, 11139-11146).

The mainstay treatments for asthma are bronchodilators, which relieve asthma symptoms by reducing airway constriction, and corticosteroids, which reduce inflammation. However, in severe and chronic asthmatic patients tissue remodelling in the airways leads both to disease progression and resistance to corticosteroid treatment. These poorly controlled patients account for >40% of the total cost of asthma treatment. Much has been learned about the nature of the airway epithelial damage in severe and chronic asthmatics, but a clinically effective means for directly targeting this problem has not previously been available.

Characteristics of such damage are a highly abnormal bronchial epithelium with structural changes involving separation of columnar cells from their basal attachments. Beneath this damaged structure, there is an increased number of subepithelial myofibroblasts that deposit interstitial collagens causing thickening and increased density of the subepithelial basement membrane. In asthmatic patients exhibiting extensive bronchial epithelial damage, such epithelium expresses markers of growth arrest (Puddicombe et al., (2003) Am. J. Respir. Cell Mol. Biol. 28, 61-68) and there is little evidence of proliferation to restore the epithelial barrier (Demoly et al., (1994) Am. J. Respir. Crit. Care Med. 150, 214-217). Prolonged epithelial repair in chronic asthma enhances cell-cell communication within the epithelial mesenchmyal trophic unit (EMTU) leading to myofibroblast activation and propagation of remodelling responses into the submucosa (Holgate et al. (2000) J. Allergy Clin. Immunol. 105, 193-204) and Holgate & Davies (2001) The Immunologist, 8, 131-135)). Mediators within the EMTU sustain inflammation while allergic mediators (Th-2 cytokines) interact with the EMTU to enhance or amplify these responses (Richter et al. (2001) Am. J. Respir. Cell Mol. Biol. 25, 385-391; Davies et al. (2003) J. Allergy Clin. Immunol. 111, 215-225). It is thus believed that a failure in the injury-repair cycle of damaged epithelial cells plays an important role in the development of abnormal epithelial-mesenchymal interactions.

U.S. Pat. No. 5,455,226 proposes use of EGF for treatment of bronchopulmonary pathologies accompanied by lesions of the bronchial epithelium. However, the specification provides no specific direction to use EGF in treatment of asthma and EGF is not recognised as having any clinical value in treatment of asthma. Indeed, it would be expected to exacerbate existing subepithelial fibrosis in asthma. This is borne out by studies of over-expression of human transforming growth factor alpha (TGFα) in a transgenic murine model of airway epithelial injury. TGFα is a structural homologue of EGF and a ligand for the EGF receptor. In the studied model, TGFα resulted in marked airway fibrosis (Korfhagen et al., Respiratory epithelial cell expression of human transforming growth factor-alpha induces lung fibrosis in transgenic mice. J. Clin. Invest. (1994) 93, 1691-1699). The only lesions of human bronchial epithelium specifically mentioned in U.S. Pat. No. 5,455,226 are ciliated respiratory epithelial wounds arising from accidental intoxication, bronchopulmonary infections, chronic bronchitis and emphysema. Moreover, proposal to use EGF to treat such wounds relies solely on demonstration of a beneficial effect of EGF on in vitro wound models of the human respiratory epithelium which are largely dependent on epithelial cell migration rather than a defect in the ability of the epithelial cells to mount a proliferative response. Hence, such models are not good models for epithelial damage and airway remodelling in asthma patients. Furthermore, it was not previously known whether the failure in the injury-repair cycle in damaged asthmatic epithelium is due to an intrinsic defect in the ability of the asthmatic epithelial cells to mount a proliferative response or to the presence of factors such as TGFβ that act as epithelial growth antagonists, or a combination of both.

It has now been found that bronchial epithelial cells from human asthma patients require exogenous EGF for maximal proliferation in primary culture whereas proliferation of bronchial epithelial cells from non-asthmatics under the same conditions is unaffected by this growth factor. This observation provided the foundation for proposing a new strategy for targeting, or protecting from, bronchial epithelium damage in asthmatics relying on use of non-natural, recombinant analogues of EGF which exhibit selective ability to promote proliferation of such bronchial epithelial cells in comparison to airway fibroblasts. By way of example, such an EGF analogue is disclosed in Puddicombe et al. (1996) J. Biol. Chem. 271, 30392-30397. That paper discloses a chimeric growth factor in which the carboxyl terminal 11 amino acid residues of mouse EGF are replaced by the corresponding 7 amino acid residues of mouse TGFα (mEGF/TGFα₄₄₋₅₀; see FIG. 1). This chimeric growth factor was found to have a relatively low affinity for the EGF receptor (EGFR) compared with EGF and, as expected, was a poor mitogen when tested on normal, human foreskin fibroblasts. In contrast, when tested on NR6/HER cells (NR6 mouse fibroblasts transfected with the human EGFR), the chimeric growth factor was a far more potent mitogen (i.e. a superagonist) than predicted by its affinity. This superagonist activity has been observed with several genetically modified EGFR ligands and is believed to be due to repeated cycles of EGFR binding and dissociation of the low affinity ligand and to alternative trafficking of the activated EGFR (Lenferink et al. (1998) Differential endocytic routing of homo- and hetero-dimeric ErbB tyrosine kinases confers signaling superiority to receptor heterodimers. EMBO J. 17, 3385-3397; Lenferink et al. (2000) Superagonistic activation of ErbB-1 by EGF-related growth factors with enhanced association and dissociation rate constants. J. Biol. Chem. 275, 26748-53). However, the inventors recognised that the cells used to measure the superagonist response were genetically modified to express the EGFR at levels that are about 10× higher than found in normal fibroblasts. As this level of EGFR expression is characteristic of epithelial cells, the inventors reasoned that the ligand might prove a potent activator of epithelial cells and, as such, might provide the selectivity needed for an epithelial growth factor with fibrosis-sparing properties useful in treatment of asthma. The inventors have now tested this recombinant chimeric growth factor in mitogenesis assays using the H292 human bronchial epithelial cell line, an established bronchial epithelial model, and human airway fibroblasts and found that it shows about 100-fold more ability to promote DNA synthesis on bronchial epithelial cells. This chimeric growth factor, species homologues thereof, especially the human homologue thereof, and other polypeptide analogues thereof which retain the ability to preferentially promote proliferation of asthmatic bronchial epithelial cells in the presence of airway fibroblasts are therefore now proposed as therapeutics for targeting bronchial epithelium damage in asthma patients.

Keratinocyte growth factor was first identified as a growth factor with marked specificity for epithelial cells compared to fibroblasts as described in EP-B 0555205. It is thus also now extrapolated that KGF and KGF analogues will equally be useful in treating, or protecting from, bronchial epithelium damage in asthma patients. While KGF has previously been proposed for use as a therapeutic in lungs, such use did not encompass asthma. Thus, EP-B 0619370 of Amgen Inc. proposes various therapeutic uses for KGF including combating lesions in the lungs arising from smoke inhalation, emphysema and pulmonary inflammation. Treatment of such lesions was not predictive of utility of KGF, or any EGF analogue, in combating deficiency of asthmatic bronchial epithelial cells to mount a proliferative response.

Although the International Patent application, WO 99/39729, of Genentech proposes the use of heregulin (HRG, also known as neuregulin) as an agent for inducing epithelial cell growth in a variety of lung diseases including asthma, this growth factor binds to distinct receptors, namely HER3 (ErbB3) and HER4 (ErbB4) (Carraway K L, Carraway C A, Carraway K L 3rd. Roles of ErbB-3 and ErbB-4 in the physiology and pathology of the mammary gland. J. Mammary Gland Biol. Neoplasia. 1997 2 (2):187-98) and thus cannot be considered to have the same receptor specificity profile as a true EGF analogue. Furthermore, while it has been demonstrated that mesenchymal cell derived HRG stimulates epithelial cell proliferation during lung development (Damman et al. Role of neuregulin-1 beta in the developing lung. Am. J. Respir. Crit. Care Med. 2003 167 (12):1711-6), it is now known that HRG is not an epithelial specific mitogen. For example, it has activity towards vascular cells (Russell et al. Neuregulin activation of ErbB receptors in vascular endothelium leads to angiogenesis. Am. J. Physiol. 1999 277 (6 Pt 2):H2205-11), muscle cells and neuronal cells (Falls D. L. Neuregulins and the neuromuscular system: 10 years of answers and questions. J. Neurocytol. 2003 June-September; 32 (5-8):619-47). As increased vascularity in the airways has been proposed to contribute to airflow limitation in asthma patients (Hashimoto et al. Quantitative analysis of bronchial wall vascularity in the medium and small airways of patients with asthma and COPD. Chest. 2005 March 127 (3):965-72), the potential of HRG to induce angiogenesis in the airways would be an undesirable property that could limit its utility as an agent that promotes epithelial repair. It will thus be understood that the term “EGF analogue” as used herein does not extend to any heregulin.

### SUMMARY OF THE INVENTION

In one aspect, the present invention thus provides use of a growth factor which is an EGF analogue, a KGF or KGF analogue in the manufacture of a medicament for use in treating, or protecting from, bronchial epithelium damage in asthma patients, said EGF analogue targeting the EGFR and exhibiting ability to promote in said patients preferential proliferation of bronchial epithelial cells compared to airway fibroblasts. It will be understood that such preferential activity will be such that the EGF analogue can be administered to the airways in a clinically effective amount to promote epithelial repair without causing clinically problematic airway fibrosis. The EGF analogue may incorporate modifications to the polypeptide chain (e.g. PEGylation) to prolong the half-life of the administered growth factor.

In a further aspect, there is provided a method of screening a test agent for ability to promote increased proliferation of bronchial epithelial cells of asthma patients which are defective in proliferative ability compared to control bronchial epithelial cells of non-asthmatics, said method comprising
(i) culturing such bronchial epithelial cells from asthma patients in the absence of growth factor;
(ii) adding to said culture, or an identical culture, the test agent and
(iii) determining whether said test agent reduces need for exogenous EGF to promote maximal proliferation or mitogenesis compared to control bronchial epithelial cells cultured under the same conditions without addition of test agent or growth factor.

Where the test agent is a polypeptide to be tested as an EGF analogue, such a screening method will also comprise the step of determining whether the EGF analogue exhibits preferential ability to promote proliferation or mitogenesis on cultured bronchial epithelial cells of asthma patients compared to cultured airway fibroblasts of the same species. However, such screening may also be used to screen for compounds which increase endogenous growth factor production in bronchial epithelial cells of asthma patients with defective proliferative ability. Such compounds are also now envisaged as potential therapeutics for use in targeting, or protecting from, bronchial epithelium damage in asthma patients.

### BRIEF DESCRIPTION OF THE FIGURES AND SEQUENCE LISTING

FIG. 1: the secondary structure of mEGF/TGFα₄₄₋₅₀ in which the seven carboxyl terminal residues of TGFα are shown in bold. The complete sequence of this chimeric growth factor is also given in SEQ. ID. No. 1. The seven carboxyl terminal residues of mouse TGFα are set out in SEQ. ID no. 3. The mouse EGF-derived sequence is set out in SEQ. ID no. 2

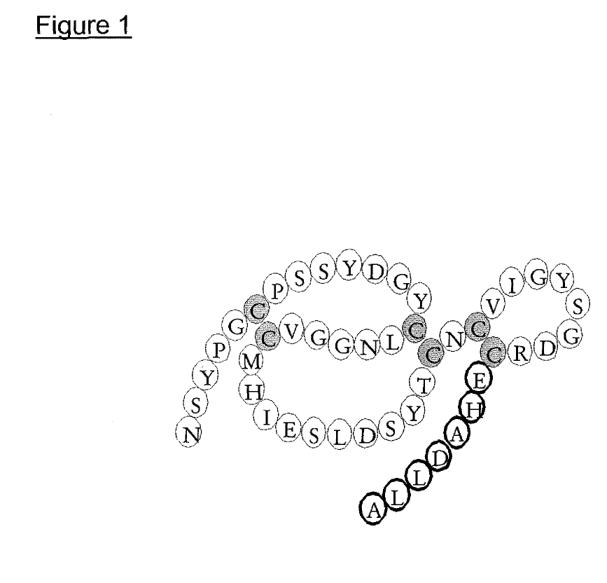

FIG. 2: the effect of EGF on normal and asthmatic bronchial epithelial cell proliferation. Primary bronchial epithelial cell cultures from 7 normal healthy human controls (a) and 10 human asthmatic subjects (b) were exposed to serum free medium (SFM) alone or in the presence of EGF as described in the Example 2. Data represents median, interquartile range and 5-95% confidence intervals. Black dots are outliers. Statistical significance was assessed using the Wilcoxon rank sum test.

FIG. 3: comparison of the mitogenic activity of EGF and mEGF/TGFα₄₄₋₅₀ towards bronchial epithelial cells (a) and bronchial fibroblasts (b). H292 bronchial epithelial cells (a) or human airway fibroblasts (b) were serum starved and then treated with increasing doses of EGF (circles) or mEGF/TGFα₄₄₋₅₀ (squares) as described in Example 2. Induction of DNA synthesis was measured 18 to 24 hours later by measuring incorporation of radioactive thymidine into acid insoluble material and scintillation counting.

### DETAILED DESCRIPTION

As indicated above, an EGF analogue for use in accordance with the invention will be such that when it is administered in a clinically effective amount to asthma patients it will promote epithelial repair without causing clinically problematic fibrosis. Such an EGF analogue may be an EGF/TGFα chimeric analogue in which C-terminal amino acid residues of an EGF, which may be wild-type or non-wild-type, are substituted by C-terminal residues of a TGFα. Thus, C-terminal amino acid residues of an EGF, especially for example the C-terminal 11 amino acid residues of a wild type or non-wild type EGF, may be replaced by the C-terminal 7 amino acid residues of a TGFα, e.g. such a human-human chimera (hEGF/hTGFα₄₄₋₅₀). Such a human chimeric growth factor (especially the human chimeric growth factor in which the C-terminal 11 amino acid residues of human EGF are substituted by the 7 amino acid residues at the C-terminus of human TGFα) is envisaged as having preferred utility in relation to human asthma sufferers, but the invention may also find applicability to non-human asthmatic animals. Moreover, functional polypeptide analogues of such chimeric growth factors may be utilised which maintain the required differential activity on bronchial epithelial cells and airway fibroblasts of asthma patients. This may be judged initially by use of conventional in vitro proliferative and/or mitogenesis assays, e.g. a mitogenesis assay as described in the exemplification employing primary cultures of confluent and quiescent bronchial epithelial cells or a human bronchial epithelial cell line such as the H292 bronchial epithelial cell line or human airway fibroblasts. Desirably in such a proliferative or mitogenesis assay a selected EGF analogue will exhibit about 10-100-fold or more activity on bronchial epithelial cells as compared to airway fibroblasts. Suitable functional analogues of an EGF/TGFα₄₄₋₅₀ chimeric growth factor, e.g. hEGF/hTGFα₄₄₋₅₀, may, have the wild-type EGF sequence substituted by a variant sequence of a known EGF analogue or, on the basis that this phenomenon can translate to other growth factors in the class, may be the wild type EGF (or a homologue of EGF) sequence with point mutations (e.g. L47A) or truncations (e.g. EGF 1-46) at key receptor binding residues that decrease EGFR binding affinity (see e.g. Groenen et al. (1994) Structure-function relationships for the EGF/TGFα family of mitogens. Growth Factors 11, 235-257) but maintain the required differential activity on bronchial epithelial cells compared to fibroblasts. Suitable EGF truncations may include EGF variants with N-terminal and/or internal deletions resulting in shorter peptide sequences which retain the required differential activity. Suitable functional analogues of chimeric growth factors as discussed above may, for example, have one or more substitutions, e.g. one or more conservative substitutions, which maintain the required differential activity on bronchial epithelial cells compared to fibroblasts, either alone or in combination with one or more deletions.

As indicated above, it is also envisaged that an EGF analogue as discussed above may be substituted by a KGF or KGF analogue for the same therapeutic purpose. Such a growth factor may be a native form of KGF such as a native form of human KGF. It may be a recombinant growth factor. The term “KGF analogue” will be understood to include any variant of native KGF which retains the required specificity for clinical use as discussed above. Such a variant may be equated with ability to stimulate DNA synthesis in quiescent BALB/MK epidermal keratinocytes by more than 500-fold while substantially lacking mitogenic activity on fibroblasts, e.g. at 5 nM exhibiting less than one-fold stimulation over background on NIH/3T3 fibroblasts. Alternatively, appropriate variants of KGF may be identified as follows and as specified in EP-A 1016716: (i) the amount of the variant that elicits maximal stimulation of BALB/MK keratinocytes elicits less than 1/50th of the maximal thymidine incorporation of NIH/3T3 fibroblasts stimulated by acidic fibroblast growth factor or basic fibroblast growth factor; or (ii) the amount of the variant that elicits maximal stimulation of BALB/MK keratinocytes elicits less than 1/10th of the maximal thymidine incorporation of NIH/3T3 fibroblasts stimulated by EGF or TGF-alpha. A number of such KGF analogues have already been described. Of particular interest in relation to the subject invention are, for example, truncated KGF analogues exhibiting increased activity on epidermal cells compared to native KGF. For example, EP-B 0706563 in the name of Chiron Corporation describes such a truncated analogue in which the N-terminal 23 amino acid residues are missing from native mature KGF. Other modifications may be incorporated in a full length mature KGF or active truncated KGF, e.g. one or more substitutions such as one or more conservative substitutions, with retention of the required activity and specificity for therapeutic use as proposed above. A KGF or KGF analogue for use in accordance with the invention may also incorporate modification to the polypeptide chain to prolong half-life upon administration as discussed above in relation to EGF analogues.

A selected growth factor as discussed above may be incorporated into any conventional form of pharmaceutical composition for airway delivery, e.g. a liquid or powder formulation for aerosol delivery as developed for other bioactive peptides (e.g. Owens et al. (2003) Alternative routes of insulin delivery. Diabet. Med. 20, 886-898; Codrons et al. (2003) Systemic delivery of parathyroid hormone (1-34) using inhalation dry powders in rats. Pharm. Sci. 92, 938-950). A suitable dosage of the growth factor may be, for example, in the range of about 0.5-50 μg daily and may include modifications as referred to above (e.g. PEGylation) which prolong the half-life of the growth factor.

In a further aspect, there is provided a method of treating, or protecting from, bronchial epithelium damage in an asthma patient, preferably a human patient, which comprises administering to the airways of said patient an EGF analogue, a KGF or KGF analogue, said EGF analogue targeting the EGFR and exhibiting ability to promote in such a patient preferential proliferation of bronchial epithelial cells compared to airway fibroblasts.

Screening Assays

As indicated above, the invention in a further aspect also extends to screening assays for agents capable of promoting increased proliferation of bronchial epithelial cells of asthma patients which are defective in such proliferation relying on determination of whether the test agent reduces the need for exogenous EGF to promote maximal proliferation or mitogenesis of such cells in vitro. In a further aspect, there is thus provided a method of screening a test agent for ability to promote increased proliferation of bronchial epithelial cells of asthma patients which are defective in proliferative ability compared to control bronchial epithelial cells of non-asthmatics, said method comprising:
(i) culturing such bronchial epithelial cells from asthma patients, preferably human asthma patients, in the absence of growth factor;
(ii) adding to said culture, or an identical culture, the test agent and
(iii) determining whether said test agent reduces the need for exogeneous EGF to promote maximal proliferation or mitogenesis compared to said control cells cultured under the same conditions without addition of test agent or growth factor.

Such screening may employ cellular proliferation or mitogenesis assays of conventional form. Where the test agent is a polypeptide to be tested as an EGF analogue, such a screening method will also comprise the step of determining whether the EGF analogue exhibits preferential proliferative or mitogenic activity on bronchial epithelial cells of asthma patients compared to that on airway fibroblasts of the same species, desirably about 10-100-fold or more activity on bronchial epithelial cells, most desirably 100-fold or more activity on bronchial epithelial cells. However, as also indicated above, such screening may also be applied to other compounds with a view to selecting compounds of potential therapeutic interest_which may increase endogenous growth factor production in bronchial epithelial cells of asthma patients and thereby improve proliferative ability and reduce airway epithelium damage.

### EXAMPLES

### Example 1

### Production of mEGF/TGFα₄₄₋₅₀ and Wild-Type mEGF

The chimeric growth factor mEGF/TGFα₄₄₋₅₀ and wild-type mouse EGF were produced in Pischia pastoris using the pPIC9 vector from Invitrogen BV, Leek, The Netherlands and characterised as previously described in Chamberlin et al., (2001) Eur. J. Biochem. 268, 6247-6255 and Puddicombe et al., (1996) J. Biol. Chem. 271, 30392-30397.

### Example 2

### Proliferation and Mitogenesis Assays

(i) Primary Epithelial Cell Cultures

Bronchial brushings were taken from non-atopic, non-asthmatic control subjects and asthmatic subjects. Volunteers were characterised according to symptoms, pulmonary function and medication. Assessment of asthma severity was in accordance with the GINA guidelines on the diagnosis and management of asthma (Bousquet (2000) Global initiative for asthma (GINA) and its objectives.

Clin Exp Allergy 30 Suppl 1:2-5). All subjects were non-smokers and were free from respiratory tract infections for a minimum of 4 weeks prior to inclusion in the study. Written informed consent was obtained from all volunteers prior to participation, and ethical approval was obtained from the Joint Ethics Committee of Southampton University Hospital Trust. Subject details are shown in Table 1. All subjects were tested for atopy using a panel of common aero-allergens and serum IgE levels were measured by standard enzyme linked immunosorbent assay (ELISA). Airway hyper-responsiveness was assessed by histamine inhalation challenge and expressed as PC₂₀ (the cumulative dose of histamine required to produce a fall in Forced Expiratory Volume in 1 second [FEV₁] by 20% from baseline).

TABLE 1
Characteristics of the volunteers that provided bronchial
brushings for primary bronchial epithelial cell culture

| | | | FEV₁ % | PC₂₀ |
|---|---|---|---|---|
| Subject | Sex | Age | predicted | (mg/ml) |
| Normals | | | | |
| 1 | F | 30 | 105 | >8 |
| 2 | F | 37 | 120 | >8 |
| 3 | M | 25 | 106 | >8 |
| 4 | M | 32 | 98 | >8 |
| 5 | M | 21 | 103 | — |
| 6 | F | 43 | 98 | >8 |
| 7 | F | 53 | 116 | >8 |
| mean | 4:3 (f:m) | 34.4 | 106.6 | >8 |
| Asthmatics | | | | |
| 1 | F | 34 | 70 | 0.16 |
| 2 | F | 28 | 85.8 | 0.29 |
| 3 | M | 21 | 91.2 | 7.97 |
| 4 | F | 34 | 63 | 0.14 |
| 5 | F | 20 | 83 | 5.7 |
| 6 | F | 20 | 75 | 1.49 |
| 7 | F | 34 | 70 | 0.16 |
| 8 | M | 21 | 76 | 0.62 |
| 9 | M | 25 | 72 | 5.10 |
| 10 | M | 58 | 77.6 | 2.51 |
| mean | 6:4 (f:m) | 29.5 | 76.4 | 2.41 |

Bronchial brushings were obtained by bronchoscopy using a fibreoptic bronchoscope (Olympus FB-20D, Tokyo, Japan) in accordance with standard published guidelines (Hurd S. Z. (1991) J. Allergy Clin. Immunol. 88, 808-814). Bronchial epithelial cells were obtained using a standard sterile single-sheathed nylon cytology brush (Olympus BC 9C-26101; Tokyo, Japan). This was passed by direct vision via the bronchoscope channel into the lower airways and five to six consecutive brushings were sampled from the bronchial mucosa of the second and third generation bronchi. Cells were harvested into 5 mls sterile phosphate-buffered saline (PBS) after each brushing. On completion of the procedure, 5 mls RPMI with 10% fetal bovine serum (FBS) were added and the sample was centrifuged at 150×g for five minutes to pellet the cell suspension. Epithelial cell purity was assessed by performing differential cell counts on cytospins of the harvested cell suspension.

Primary cultures were established by seeding freshly brushed bronchial epithelial cells into culture dishes containing 3 mls of serum-free hormonally-supplemented Bronchial Epithelium Growth Medium (BEGM; Clonetics, San Diego, Calif.) supplemented with 50 IU/ml penicillin and 50 μg/ml streptomycin (Lordan et al. (2002) J. Immunol. 169, 407-414). When confluent, the cells were passaged (p1) using trypsin and were allowed to further expand until used for experimentation at passage 2 or 3. Control experiments confirmed that there was no significant difference between the responses of the cells at p2 or p3. Viability was assessed by exclusion of trypan blue dye and the epithelial nature of cells assessed by immunohistochemical staining of cultures grown on culture chamber slides (Nunc, Labtek II eight well chamber slides, Life Technologies Ltd., Scotland) using a pan-cytokeratin antibody as well as antibodies specific for cytokeratin 13 (CK13) and CK18.

(ii) Effect of EGF on Proliferation

Cells were prepared in 24 well trays at a seeding density of a minimum of 4×10⁴ cells/ml primary BECs. Once 70% confluent, cells were serum starved for 24 hours in BEGM containing 1% ITS and 1% BSA respectively. Cells were treated with SFM or 1.7 nM EGF for 24 hours. For each condition, cells were prepared in duplicate. Supernatants were collected from the cells at the end of each incubation, and the cells fixed in formal saline (4% (v/v) formaldehyde in 0.9% (v/v) saline solution) for at least 30 minutes at room temperature. Formal saline was then removed and the tray blotted on tissue paper to remove excess moisture. 200 μl of 1% (v/v) methylene blue (5 g methylene blue dissolved and filtered in 500 ml 10 mM borate buffer, (3.82 g borate disodium tetraborate, made to 1 litre in distilled water, pH 8.5)) was added to each well for 30 minutes. Excess dye was removed by careful washing in tap water until the water ran clear and the tray was blotted on tissue paper. The dye was then eluted by addition of 200 μl per well of 1:1 ethanol: 0.1% HCl solution (200 ml ethanol:200 ml of 0.1M HCl) and left for 30 minutes. Absorbance of the eluant was determined using a Multiskan ascent plate reader after a 1:10 dilution with 1:1 ethanol:0.1% HCl to provide an absorbance within the linear range of the plate reader. Absorbance was read using a 630 nm filter and each sample was tested in duplicate. A standard curve was generated by direct cell counting to enable cell number to be related to methylene blue readings. An A₆₃₀ of 1.0 was equivalent to 5.5×10⁵ cells/ml.

Results

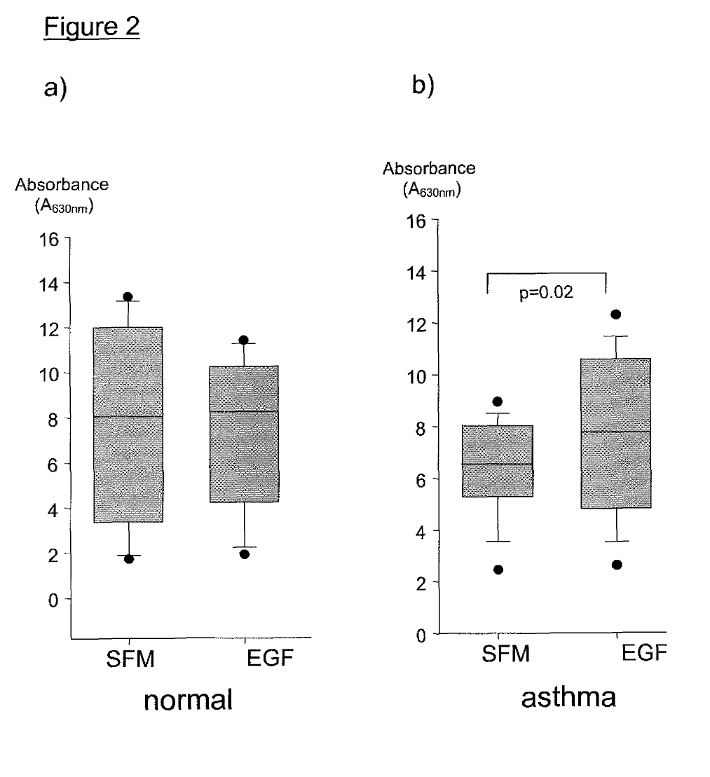

EGF treatment of primary bronchial epithelial cells from normal subjects had no effect on cell number as determined using methylene blue incorporation (see FIG. 2a). In contrast, EGF treated asthmatic cells showed a small but significant increase in cell number which approached that seen in controls (see FIG. 2b). These data indicate that the reduced proliferative rate observed in asthmatic bronchial epithelium may slow epithelial repair in response to damage and contribute to the continued disruption of the epithelial barrier and hence disease progression in asthma patients.

(ii) Mitogenesis Assays

The ability of EGF or mEGF/TGFα₄₄₋₅₀ to induce DNA synthesis in confluent and quiescent H292 bronchial epithelial cells or primary airway fibroblasts was measured in a modification of a standard mitogenesis assay (Puddicombe et al. (2000) FASEB J. 14, 1362-1374; Puddicombe et al. (1996) J. Biol. Chem 271, 30392-30397). In brief, cells were grown to confluence in 96-well opaque cell culture trays in RPMI/10% FBS and rendered quiescent by serum reduction. Growth factors were added to the cells in mitogenesis assay buffer and DNA synthesis was determined 18 h later by incorporation of ³H thymidine or the thymidine analogue, [¹²⁵I]UdR over a 2 h pulse period. The cells were fixed and washed with 5% trichloroacetic acid followed by methanol. After drying, acid-insoluble material was dissolved in 40 μl/well of 0.2M NaOH and radioactivity determined on a Topcount Scintillation counter (Canberra Packard) after addition of 150 μl of Microscint-40 (Canberra Packard, Pangbourne, Berks, RG8 7AN) to each well.

Results

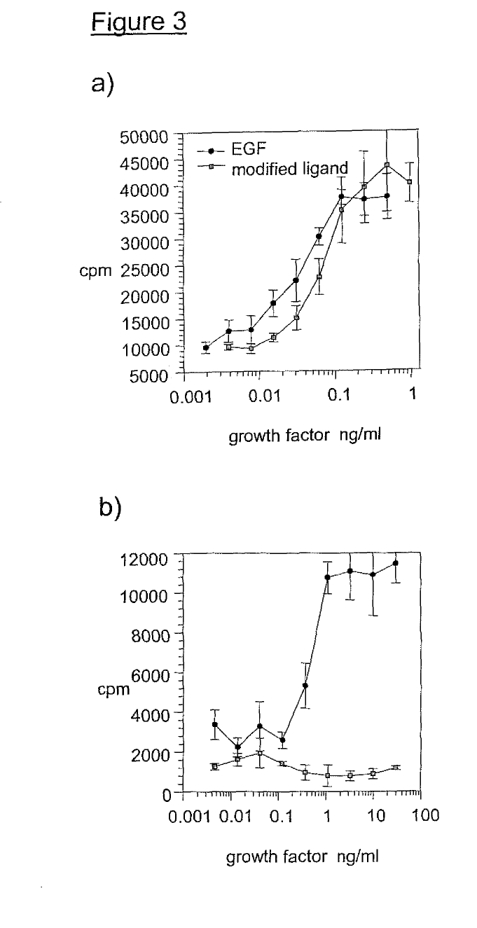

As shown in FIG. 3a, there was no difference in the ability of wild-type EGF and the chimeric growth factor to stimulate a mitogenic response on bronchial epithelial cells. However, there was marked difference in their mitogenic properties towards bronchial fibroblasts (see FIG. 3b). It is thus extrapolated that EGF/TGFα₄₄₋₅₀ chimeric growth factors and functional analogues thereof represent means for accelerating bronchial epithelium repair in asthmatics.

1-12. (canceled)

13. A method of treating, or protecting from, bronchial epithelium damage in an asthma patient which comprises administering to said patient a growth factor which is an EGF analogue, a KGF or KGF analogue, said EGF analogue targeting the EGF receptor and exhibiting ability to promote in such a patient preferential proliferation of bronchial epithelial cells compared to airway fibroblasts.

14. The method as claimed in claim 13 wherein said growth factor is administered to the airways by airway delivery.

15. The method according to claim 13 wherein said patient is a human asthma patient.

16. The method as claimed in claim 13 wherein said growth factor is an EGF/TGFα₄₄₋₅₀ chimeric analogue in which C-terminal amino acid residues of a wild-type or non-wild-type EGF are substituted by the 7 amino acid residues at the C-terminus of a TGFα.

17. The method as claimed in claim 16 wherein said analogue is an EGF/TGFα₄₄₋₅₀ chimeric analogue in which the C-terminal 11 amino acid residues of a wild-type EGF are substituted by the 7 amino acid residues at the C-terminus of a TGFα.

18. The method as claimed in claim 15 wherein said growth factor is the chimeric growth factor hEGF/TGFα₄₄₋₅₀ in which the 11 C-terminal amino acid residues of human EGF are substituted by the 7 amino acid residues at the C-terminus of human TGFα, or a functional analogue of said chimeric growth factor.

19. The method as claimed in claim 13 wherein said growth factor is a KGF or KGF analogue.

20. A method of screening a test agent for ability to promote increased proliferation of bronchial epithelial cells of asthma patients which are defective in proliferative ability compared to control bronchial epithelial cells of non-asthmatics, said method comprising:
(i) culturing such bronchial epithelial cells from asthma patients in the absence of growth factor;
(ii) adding to said culture, or an identical culture, the test agent and
(iii) determining whether said test agent reduces the need for exogenous EGF to promote maximal proliferation or mitogenesis compared to control bronchial epithelial cells cultured under the same conditions without addition of test agent or growth factor.

21. The method as claimed in claim 20 wherein said determining is by means of mitogenesis assay in which DNA synthesis is determined.

22. The method as claimed in claim 20 wherein said cells are human cells.

23. The method as claimed in claim 20 wherein said test agent is an EGF analogue and which further comprises the step of determining whether said analogue exhibits preferential ability to promote proliferation or mitogenesis on cultured bronchial epithelial cells of asthma patients compared to cultured airway fibroblasts of the same species.

24. The method as claimed in claim 20 wherein said test agent is other than an EGF analogue or EGF analogue containing composition.
